(19) United States
(12) Patent Application Publication
(10) Pub. No.: US 2016/0067369 A1
(43) Pub. Date: Mar. 10, 2016

(54) Cancer protection device

(71) Applicant: Asghar Zubair Muhammad, Toronto (CA)

(72) Inventor: Asghar Zubair Muhammad, Toronto (CA)

(21) Appl. No.: 14/842,009

(22) Filed: Sep. 1, 2015

Related U.S. Application Data
(60) Provisional application No. 62/046,073, filed on Sep. 4, 2014.

(51) Int. Cl. A61L 9/22; B01D 46/00

(52) U.S. Cl. CPC A61L 9/22; B01D 46/0026

(57) ABSTRACT

A natural base Himalayan Salt ionizer, which neutralizes and inhibits hazardous Electromagnetic field radiations EMF from our surrounding atmosphere. EMF is one of the biggest sources of Cancer cell growth and Melatonin loss. Himalayan salt has 84 essential minerals and trace elements, which support our body and respiratory system to breathe fresh air in any environment to improve our health quality. It reduces and inhibits many illnesses such as Asthma, Insomnia, migraines and headaches, allergies, dust particles, bacteria, fungus and pollutant etc. etc. . . . It also increases immunity, maintain normal blood pressure, enhances blood flow, reduces stress, depression and also enhances sexual abilities, so every time when heater turns on, the heating lights o PTC heating element gets hot, It produces heat which touches to the Salt rock crystals and it emits and produces negative ions. which bond with positive ions via ionization, neutralizing excess EMFs in the surrounding environment. Negative ions are beneficial for us because it neutralizes positive harmful ions EMF. EMF come through different sources e.g Cell phones, TV, Computers, Microwave ovens and many others electrical and mechanical devices. Natural Himalayan salt heaters/Air conditioners have ability to neutralize and purify the polluted air by increasing negative ions, which bond with positive ions via ionization, neutralizing excess EMFs in the surrounding environment. Simply put, the heat of the PTC heating elements or light warms the salt, which attracts humidity and creates a buildup of negative ions. Excessive positive ions bond with these negative ions (ionization), neutralizing the EMF waves in the atmosphere.

### CROSS-REFERENCE TO RELATED APPLICATIONS

The present application relates and claims priority to U.S. provisional patent application No. 62/046,073 filed on Sep. 4, 2014.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

1. Field of the Invention

The present invention relates to the devices of protection from cancer.

2. Description of the Related Art

Currently we are living and breathing in the world, where almost everyone's surrounded by the invisible harmful radiations EMF, which is one of the biggest sources of causing cancer. Ironically, the world don't have any device which can naturally reduce and eliminate the harmful effects of radiations coming from different electronic or semi electronic sources or appliances, which we come across in everyday life e.g. Cell phones, Computers, Microwaves oven, TVs, Cars, Radios. or any other electrical and mechanical devices. There are so many electrical devices available in the market for purifying air pollution and humidifiers, but none of them have capacity to inhibit or neutralize EMF through natural way or sources and also prevent us from cancer cell growth or Melatonin loss. The natural ionization of a Himalayan crystal salt cannot be compared to an ionizer. An ionizer can only have an ionization effect. However, Himalayan salt crystal ionizer and purifier has many advantages in many aspects. In this modern time where we are surrounded by hazardous invisible radiations, our body immensely need natural Himalayan crystal salt. It contains all the elements of which the human body is comprised. From the periodic table of elements we are familiar with 94 natural elements, all of these elements can be found in crystal salt. Hence, crystal salt contains all natural 84 minerals and trace elements out of 94 that are found in the human. Our body enormously need these elements to protect ourselves from harmful radiations and toxins. The world's truly needs something, which can enhance human health quality as well as eliminate and neutralize Electromagnetic field radiations EMF, through natural process. Negative ions are found naturally in nature, before Thunderstorms, at Himalayan salt rock mountains, at Waterfalls and wherever Nature is most pure.

### BRIEF SUMMARY OF THE INVENTION

The main aspect of the present invention is to control and protect our body from cancer cell growth and prevent melatonin loss. This invention has ability to protect our body from harmful and hazardous EMF radiations. Another aspect of the present invention is, It reduces and cures respiratory illnesses and other sickness and allergies, such as Asthma, sinus infections, Headaches/migraines and airborne allergies. The present invention increases blood flow and also maintains normal blood pressure, it also purifies polluted air and general atmosphere, it eliminates and kills airborne microorganisms e.g Fungi Molds, Bacteria, Allergens and any polluted air or dust particles. The importance of the present invention, it is the only device which contains all the elements of which the human body is comprised. From the periodic table of elements we are familiar with 94 natural elements, all of these elements can be found in Himalayan crystal salt. Hence, crystal salt contains all natural 84 minerals and trace elements out of 94 that are found in the human.

### DESCRIPTION OF THE DRAWINGS

For a more complete understanding of the present invention, reference is now made to the following descriptions:

FIG. 1 Is the body of the device and show the external view of the device.

FIG. 2 Is a diagrammatic overview of the present invention.

### REFERENCE NUMERALS IN THE DRAWINGS

For a more complete understanding of the present invention parts, reference is now made to the following descriptions:
1. Polluted and unpurified air.
2. Pre-Air Filter.
3. Electric Fan.
4. Hepa complete filter.
5. PTC Ceramic heating element” (Positive temperature coefficient).
6. Himalayan salt rock bricks.
7. Compact fluorescent light bulb/tube (CFL).
8. Negative ions and minerals.
9. Neutralize and inhibit harmful EMF.
10. Front chamber/case.
11. Purified ionic clean air and minerals.
12. The back case.
13. Electric Plug.
14. Electricity power socket.
15. Body.
16. Front case grill.
17. Turn on/off power button.
18. LCD display screen and control panel.
19. Remote control.
20. The back case grill.

### DETAILED DESCRIPTION OF THE INVENTION

The following description and embodiment of the present invention will open the new horizons in the world of cancer treatment and its preventions. In this modern society where health issues, radiations and Cancer become our basic concerns, this present embodiment of invention will help us to prevent from the harmful effects of “Electromagnetic radiations EMF and also inhibit its harmful activities. EMF is one of the basic causes of Cancer, present embodiment of invention will also prevent from cancer cell growth and melatonin loss. Present invention has some other potential benefits as well, such as it has excellent ability to treat and cures Asthma, reduces stress level/depression, headaches and migraines, Relieves Insomnia, relieves all Sinus symptoms and allergies, alert mind and body, increases sexual performance, etc.

The main theme of this invention, basically It came in my mind one day, when I was sitting inside my house and I had bad sinus, severe headache and sneezing, in the very cold weather in Canada, I was living in the basement, where we usually come across with molds, fungi, bacteria and other unseen micro organism, due to less ventilation and damp environment, so, I was sitting in the front of my portable space heater, and on the other side a humidifier was on, all of a sudden the idea popped up in my mind, I wondered what if we had something which can eliminates or reduces the potency of these molds fungi and bacterial activities and could protect our body from the harmful effects of electromagnetic radiations field and micro organisms through natural process. I knew that already, there is something present in the mother earth, which can treat or eliminate all these negative harmful effects naturally, the reason why, because I had spent a huge time, in the place where Himalayan Rock salt mountains are situated. I have observed already, the power and magic of himalayan salt, so I decided to invent something which can be helpful for the whole mankind to have a better and healthy life naturally, so that, I did some research on my own, then I personally went to the Himalayan region in Pakistan (Kohata) to find out the truth. I interviewed and visited local medical centers and also seen patients, where they came for treatment and cure of their diseases and illnesses from the different regions. I found them they were suffering in several breathing respiratory and sinus illnesses and they were recovering with good health by having and using himalayan salt rocks stuffs in their everyday lives, I also interviewed Doctors/Physicians and researchers, I also visited the site of salt mountains tunnels thoroughly, then I gave some free samples of himalayan salt lamps to few patients. They reported me and found spectacular positive and incredible results and also a great change in their lives, health and living style.

so I decided to invent Ionic natural Neutralizer Heater, air purifier Ionizer and air conditioner for general use, by mounting and using himalayan rock salt as for healing purpose inside the heater or air conditioner appliances for general public around the world. I personally tried, as well on other people, they found excellent results. The basic reason of my embodiment to invent a device where everyone can get access to have pure air and less microbial effects, but the most important and great reason to invent this device is, this device inhibits and neutralizes the harmful and hazardous effects of radiations EMF. Himalayan salt rock naturally protect our body to have less chances to get cancer, because it inhibits cancer cell growth and prevent melatonin loss. Melatonin has been shown in many clinical studies around the world to inhibit cancer cell growth. Low levels of Melatonin have also been shown in studies to stimulate cancer cell growth.

I am usually at my computer for 2-3 hours at a time. I always felt drained of energy, lethargic and had what I described as a dullish sore head. I started using my own invention prototype Himalayan Crystal Salt Heater next to my computer. I could not believe the difference! No tiredness, lethargy, no headache, and I literally feel the energy from it. I never use my computer now without my himalayan salt heater (the present invention).

Recent studies have linked EMFs to increased leukemia, lymphoma, brain cancer, melanoma, breast cancer, miscarriage, birth defects, suicide, and most recently, Alzheimer's disease. Researchers have discovered that EMFs can suppress the pineal glands, secretion of melatonin, a hormone which controls our wake/sleep cycle, moods and task performance. Depressed melatonin levels are associated with mood changes, depression and psychiatric disorders. Melatonin also plays a critical cancer role, by increasing the phytotoxicity of the body's natural killer lymphocytes. Suppression of pineal gland function has been implicated in the etiology of breast, ovarian, prostate and melanoma cancers. Other studies have linked EMFs to decreased production of enzymes called “protein kinases” in human lymphocyte cells. This also indicates that EMFs can suppress the immune system, It is therefore important for the cancer patient to limit exposure to EMF radiation, and from regular sources of EMF radiation, including the household computer, TV, cell phones, microwave oven, electric blanket and cordless telephone, in order to maintain a strong immune system.

The basic concept is to invent this device is to protect ourselves from the harmful radiations, Electro-magnetic field radiations (EMF), this device will neutralize the harmful EMF by emitting negative ions from the himalayan salt, so it will be highly helpful and beneficial for us to have very less chances to get cancer, because this device will inhibits harmful EMF which is one of the biggest sources of causing cancer, the present invention will also inhibit pollutants and microorganisms from our surrounding environment. It will provide non polluted purified and clean air.

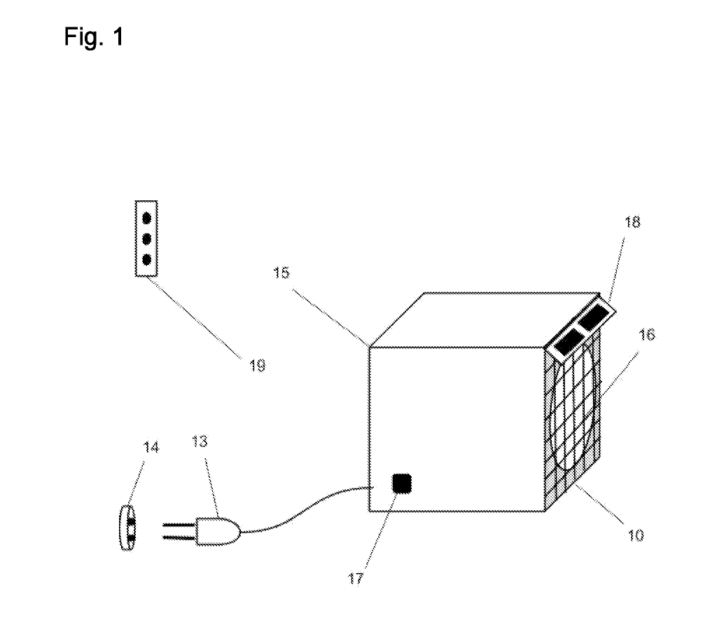

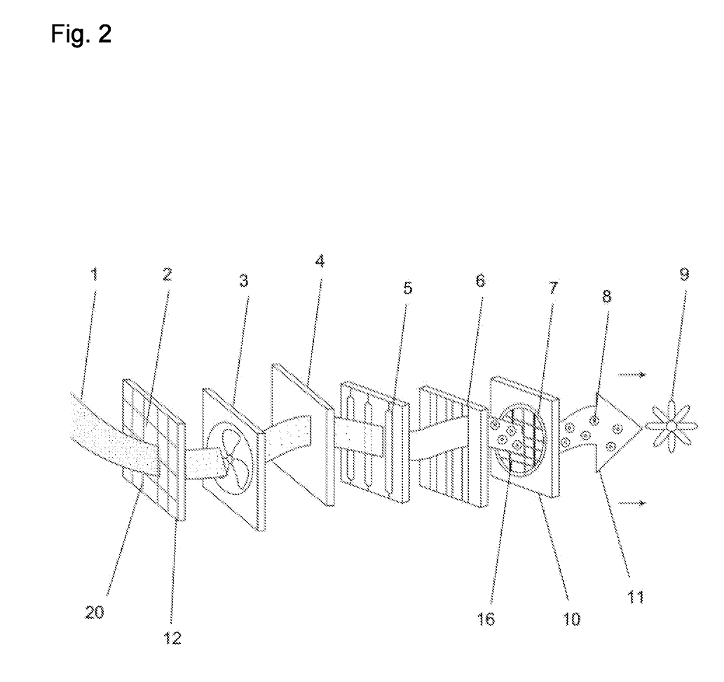

Referring to FIG. 1 this is the body 15 of the device where we can see the front case grill 16 that is included in the front chamber/case 10, the front case grill 16 to blow internal air outside in the environment also the front case grill 16 is for safety and protection purposes so it can protect anyone from the electric fan and the other components inside the device, so we can run the present invention we have to connect electric plug 13 to the electricity power socket 14 then use the turn on/off power button 17 to turn the device on. This Device has ability to go up to 1500 Watt. The present invention has a LCD display screen and control panel 18, this LCD display screen and control panel 18 consists of On & off button and other buttons like temperature low/High (Digital thermostat for setting temperature or analog knob for manually adjustments, for light (CFL) turn on/off and fan on/off, there is a remote control 19 integrates with the device LCD display screen and control panel 18, the device can be operated and controlled manually or by the remote control 19. The remote control 19 consists of a processor, Integrated circuit and Infrared unit. In the remote control 19 we can set timing turn off to control the device runtime. Referring to FIG. 2 the diagrammatic overview of the present invention where we can see how the present invention works, it starts from the left, it starts with the polluted and unpurified air 1 in our surrounding environment, the polluted and unpurified air 1 enters into the present invention through Pre-Air Filter 2 and the back case grill 20 included in the back case 12 of the present invention. The first object will be a Removable Pre-Air Filter 2, it mounts inside the back case 12, which performs obstructions and blocking of dust particles, hairs and other pollutants, present in the air and surrounding environment, also the back case grill 20 included in the back case 12 helps to get Air inside the device through electric fan 3 when electric fan 3 starts sucking air, also the back case grill 20 for safety and protection purposes so it can protect anyone from the electric fan 3 and the other components inside the device. The second object will be an electric fan 3, it is for sucking air which goes inside the present invention and also pushes partially purified air to the next level which is hepa complete filter 4. The air enters and passes through the hepa filter 4. The function of hepa filter 4 is to purify and filter air completely without any toxins, microorganisms and any other pollutants, so then filtered and purified air will hit and touch to the PTC Ceramic heating element (Positive temperature coefficient) 5, PTC Ceramic heating element” (Positive temperature coefficient) 5 for heating purpose, which will produce heat waves. This heat waves will touch and hit to the Himalayan salt rock bricks 6, then, it will generate and produce negative ions and minerals 8. The Ionic purified warm air will blow through the front chamber or front case 10 of the device to our surrounding environment, then It will start neutralizing and inhibiting harmful EMF 9 in atmosphere via ionization. Then final front case 10, mounted with Health Canada government recommended small compact fluorescent light bulbs CFL 7, facing towards himalayan salt bricks 6. why Compact fluorescent light bulb/tube (CFL) 7 lights inside the front case 10 of the device, because the rays and the heat of the compact fluorescent bulb/tube (CFL) 7 light will help to emit negative ions and minerals 8 from the himalayan salt bricks 6. especially in the case when we don't need to have electric fan's air on or don't need any heat. So, then we can only turn on the Compact fluorescent light bulb/tube (CFL)s 7 lights. When needed. It will work like a natural Ionic power light Lamp, neutralize and inhibit harmful EMF 9 inhibitor and reducer. We can use this Lamp in all seasons. It will produce and disperse negative ions and minerals 8 throughout the year and will neutralize and inhibit harmful EMF 9 through natural way, by following this process we can protect ourselves from the harmful radiations and have purified ionic clean air and minerals 11. The present invention have the ability to neutralize and inhibit harmful EMF 9 and purify polluted air 1 by increasing negative ions and minerals 8, which bond with positive ions via ionization, neutralizing excess EMFs 9 in the surrounding environment. Simply put the heat of the light warms the salt, which attracts humidity and creates a buildup of negative ions and minerals 8. Excessive positive ions bond with these negative ions and minerals 8 (ionization), neutralizing the EMF 9 waves in the atmosphere.

1. A device to purify air and inhibits excess harmful EMF radiations in the surrounding environment to protect from cancer, it purifies air and neutralizes harmful effects of EMF radiations through ionization, this device prevent us from Cancer cell growth and melatonin loss, this device reduces and cures respiratory illnesses and other sickness and allergies, such as Asthma, sinus infections, headaches/migraines and airborne allergies, the device increases blood flow and also maintains normal blood pressure, the device purifies polluted air and general atmosphere, it eliminates and kills airborne microorganisms e.g Fungi Molds, Bacteria, Allergens and any polluted air or dust particles, the device contains all the elements of which the human body is comprised, from the periodic table of elements we are familiar with 94 natural elements, all of these elements can be found in himalayan crystal salt, hence, the crystal salt contains all natural 84 minerals and trace elements out of 94 natural elements that are found in the human, the device will prevent us from the harmful effects of Electromagnetic field radiations EMF and also inhibit its harmful activities, the device will prevent from cancer cell growth and melatonin loss, the device is an Ionic natural neutralizer heater, air purifier Ionizer and air conditioner for general use, by mounting and using himalayan rock salt as for healing purpose inside the heater or air conditioner appliances for general public around the world, this device will help everyone to get access to have pure air and less microbial effects, this device inhibits and neutralizes the harmful and hazardous effects of radiations EMF, himalayan salt rock naturally protect our body to have less chances to get cancer, because it inhibits cancer cell growth and prevent melatonin loss, melatonin has been shown in many clinical studies around the world to inhibit cancer cell growth, low levels of melatonin have also been shown in studies to stimulate cancer cell growth, to protect ourselves from the harmful radiations, Electro-magnetic radiations (EMF), this device will neutralize the harmful EMF by emitting negative ions from the himalayan salt, so it will be highly helpful and beneficial for us to have very less chances to get cancer, because this device will inhibits harmful EMF which is one of the biggest sources of causing cancer, this device will also inhibit pollutants and microorganisms from our surrounding environment, the device will provide non polluted purified and clean air.

2. A device to purify air and inhibits excess harmful EMF radiations in the surrounding environment to protect from cancer comprising:
pre-air filter;
electric fan;
hepa complete filter;
PTC ceramic heating element (Positive temperature coefficient);
himalayan salt rock bricks;
compact fluorescent light bulb/tube (CFL);
front chamber/case;
the back case;
electric plug;
electricity power socket;
body;
front case grill;
turn on/off button;
LCD display screen and control panel;
remote control; and
the back case grill.

3. A device to purify air and inhibits excess harmful EMF radiations in the surrounding environment to protect from cancer comprising:
the first step to run the device we have to connect the electric plug to the electricity power socket then use the turn on/off power button to turn the device on, the present invention has ability to go up to 1500 Watt, the present invention has a LCD display screen and control panel, this LCD display screen and control panel consists of On & off button and other buttons like temperature low/High, digital thermostat for setting temperature or analog knob for manually adjustments, light (CFL) turn on/off and electric fan on/off, there is a remote control integrates with the device LCD display screen and control panel, the device can be operated and controlled manually or by the remote control, the remote control consists of a processor, Integrated circuit and Infrared unit, in the remote control we can set timing turn off to control the device runtime, the polluted and unpurified air enters into the present invention through pre-air filter and the back case grill included in the back case/grills of the present invention, the first object will be a pre-air filter, it mounts inside the back case which performs obstructions and blocking of dust particles, hairs and other pollutants, present in the air, the second object will be a electric fan, it is for sucking air which goes inside the present invention and also pushes partially purified air to the next level which is hepa complete filter, also the back case grill included in the back case helps to get air inside the device through electric fan when electric fan starts sucking air, also the back case grill for safety and protection purposes so it can protect anyone from the electric fan and the other components inside the device, the air enters and passes through the hepa filter, the function of hepa filter is to purify and filter air completely without any toxins, microorganisms and any other pollutants, so then filtered and purified air hits and touches to the, PTC Ceramic heating element” (Positive temperature coefficient) which produces heat waves, this heat waves touches and hits to the himalayan salt rock bricks, then, it generates and produces negative ions and minerals, the Ionic purified warm air blows through the front chamber or front case of the device to our surrounding environment, then It starts neutralizing and inhibiting harmful EMF in atmosphere via ionization, then final front case, mounted with health Canada government recommended small compact fluorescent bulbs CFL, facing towards himalayan salt bricks, why compact fluorescent bulb/tube (CFL) lights inside the front case of the device, because the rays and the heat of the compact fluorescent bulb/tube (CFL) light helps to emit negative ions and minerals from the himalayan salt bricks, especially in the case when we don't need to have fan's air on or don't need any heat, so, then we can only turn on the compact fluorescent bulb/tube (CFL)s lights, when needed, it works like a natural Ionic power light Lamp, neutralize and inhibit harmful EMF inhibitor and reducer, this lamp can be used in all seasons, it produces and disperses negative ions and minerals throughout the year and neutralize and inhibit harmful EMF through natural way, by following this process we can protect ourselves from the harmful EMF radiations and have purified ionic clean air and minerals, the present invention have the ability to neutralize and inhibit harmful EMF and purify polluted air by increasing negative ions and minerals, which bond with positive ions via ionization, neutralizing excess EMFs in the surrounding environment, simply put the heat of the light warms the salt, which attracts humidity and creates a buildup of negative ions and minerals, excessive positive ions bond with these negative ions and minerals (ionization), neutralizing the EMF waves in the atmosphere.
